(19) United States
(12) Patent Application Publication
(10) Pub. No.: US 2014/0276359 A1
(43) Pub. Date: Sep. 18, 2014

(54) APPARATUS AND METHOD FOR TISSUE REJUVENATION

(71) Applicant: PLUM SYSTEMS CO., New York, NY (US)

(72) Inventor: Alexandra Alvarez, New York, NY (US)

(73) Assignee: Plum Systems Co., New York, NY (US)

(21) Appl. No.: 13/833,902

(22) Filed: Mar. 15, 2013

(51) Int. Cl. A61M 5/32

(52) U.S. Cl. CPC A61M 5/3295
USPC 604/21; 604/26; 604/173

(57) ABSTRACT

A method and an apparatus of applying carboxytherapy to a subject is disclosed. The method comprises the steps of: contacting a target body surface of the subject with a treatment device comprising a plurality of hollow needles attached to a contact surface of a housing and a CO₂ source in fluid communication with at least one of the plurality of hollow needles; applying pressure to the housing such that one or more of the plurality of hollow needles penetrate an epidermis or an outermost layer of cell in the target body surface; applying a therapeutic amount of CO₂ to the subject through said plurality of hollow needles; and removing the plurality of hollow needles from said target body surface. A method of applying carboxytherapy to improve skin conditions is also disclosed.

### FIELD

This application generally relates to tissue repair in human subjects. In particular, the invention relates to devices and methods for localized administration of carbon dioxide.

### BACKGROUND

Carboxytherapy, also known as carbon dioxide (CO₂) therapy or CDT is a safe and effective therapy that can improve the appearance of treated skin. Outside of the US, carboxytherapy has found applications in the reduction of skin irregularities, wrinkle reduction and as a complementary treatment to other forms of aesthetic and therapeutic treatments, such as liposuction.

While carboxytherapy has been studied in academic settings for some medical conditions, the exact mechanism of action of carboxytherapy is not well understood. Accordingly, drug and device-based innovations have been the main pathway to skin treatment. However, as patients and medical professionals begin to shy away from purely medical means of treatments carboxytherapy provides a more “natural” alternative.

As currently applied, carboxytherapy is a non-surgical method, but which requires a treatment by a medical professional. CO₂ is inserted using needles into the subcutaneous tissue through very small needles, for example 30G needles. From the injection point, the CO₂ diffuses into adjacent tissues. While carboxytherapy uses innocuous amounts of inert CO₂ gas, the use of needles as a the mode of application requires that a medical professional to apply the treatment.

At present, application methods require the medical professional to insert single needles into the patient for treatment. For small areas of treatment such, as the eye and lip areas of patients, such individual insertion is safe and effective. However, for treatment of large areas, individual treatment is unduly rigorous and time consuming. The benefits of carboxytherapy for treating a wide range of dermatological conditions have therefore not been fully obtained by patients.

### SUMMARY

One aspect of the present invention relates to a method of applying carboxytherapy to a subject in need of such treatment, comprising the steps of: contacting a target body surface of said subject with a treatment device comprising: a plurality of hollow needles attached to a contact surface of a housing; and a CO₂ source in fluid communication with at least one of said plurality of hollow needles; applying pressure to said housing such that one or more of the plurality of hollow needles penetrate an epidermis or an outermost layer of cell in said target body surface; applying a therapeutic amount of CO₂ to said subject through said plurality of hollow needles; and removing said plurality of hollow needles from said target body surface.

Another aspect of the present invention relates to a method for treating hair loss in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

Another aspect of the present invention relates to a method for treating a skin condition in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

Another aspect of the present invention relates to a method for promoting wound healing in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

Yet another aspect of the present invention relates to a disposable skin treatment device, comprising a plurality of hollow needles attached to a surface of a base, wherein said hollow needles having diameters in the range of 0.1-0.5 mm and lengths in the range of 0.4-2.5 mm and wherein said base is attachable to a handheld device to form a fluid communication between at least one of said plurality of hollow needles; wherein at least one of said plurality of hollow needles comprises a sharp end capable of penetrating human epidermis under hand pressure.

### BRIEF DESCRIPTION OF THE DRAWINGS

The present invention can be better understood by reference to the following drawings. The drawings are merely exemplary to illustrate certain features that may be used singularly or in combination with other features and the present invention should not be limited to the embodiments shown.

FIG. 1 depicts the exterior contact surface of an exemplary application device.

FIG. 2 shows the interior of the contact surface of FIG. 1.

FIG. 3 shows the valves of the exemplary application device of FIG. 1.

FIG. 4 shows the handle and controls of the exemplary application device of FIG. 1.

FIG. 5 is a side view of the exemplary application device of FIG. 1 showing the CO₂ supply tube.

FIG. 6 shows another exemplary embodiment of an application device having microneedles affixed thereto.

FIG. 7 is an illustration of the penetration of the skin by the microneedles of the device.

FIG. 8 depicts an exemplary configuration for the microneedles in the base element.

FIG. 9 depicts another exemplary configuration for the microneedles in the base element.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description and claims, the singular forms “a,” “an” and “the” include plurals unless the context clearly dictates otherwise. The present invention provides improved methods and devices to apply carboxytherapy to an area of a patient. The device includes a treatment device fittable with a microneedle/base element where a plurality of the microneedles are in fluid communication with a source of CO₂. To apply the CO₂, the device is placed on the surface of the skin or scalp of the patient as described below.

As used herein, “administering to the skin in need of such treatment” means contacting (e.g., by use of the hands or an applicator) the area of skin in need such treatment. These features may be present on the face, such as under or adjacent the eyes, nose, forehead, cheeks, jowls, and neck, as well as other areas of the body such as the arms, chest, back, shoulder, belly (e.g., stretch marks), and legs (e.g., cellulite).

The term “treating” or “treatment” of a skin disorder means the treatment (e.g., complete or partial reduction or elimination of symptoms and/or cure) and/or prevention or inhibition of the skin disorder.

As used herein, the term “skin disorder” or “skin condition” means a disease, disorder, or defect of the skin.

### Microneedles

As used herein, the terms “needle” and “microneedle” refer to a piercing element suitable for passing gas therethrough that is also suitable to pierce the skin of a patient or subject in accordance with a carboxytherapy method. The terms are interchangeable unless the context clearly dictates otherwise.

Piercing of the skin service disrupts the stratum corneum. By only piercing the stratum corneum and/or other layers of the epidermis, the patient typically does not feel pain, trauma (e.g., bleeding and swelling), and/or other discomfort. Carboxytherapy allows CO₂ gas to be transported through the disrupted skin to provide the therapeutic benefits. The diameter of the microneedles used to both pierce a patient's skin and to apply carboxytherapy in the present invention must be of a suitable gauge to allow gas to flow therethrough and at the same time suitable piercing of a variety of skin types and thicknesses, but not so large so as to cause discomfort to the patient when the needles pierce the skin.

The term “plurality of microneedles” means a collection of microneedles arranged for use in the device and methods herein. Such plurality of microneedles must be securably attached to the base material so as to allow suitable insertion into the skin of a patient and also to allow application of other forms of treatment. Examples of suitable materials for use as the microneedles herein can include one or more of metals and metal alloys such as, for example, stainless steel, gold, iron, steel, tin, zinc, copper, platinum, aluminum, germanium, zirconium, titanium and titanium alloys containing molybdenum and chromium, metals or non-metals plated with, gold, rhodium, iridium, titanium, platinum, silver, silver halides, and alloys of these or other metals.

When considered individually, the microneedles used in the present invention can have substantially straight or substantially tapered shafts. The diameter of the microneedles can be larger at the base end of the microneedle to taper to a point at the end distal the base. The microneedles can be circular or semi-circular or any other suitable cross-sectional shape. For example, the cross-section of the microneedle can be polygonal (e.g., star-shaped, square, triangular, rectangular), oblong, or another shape. However, whatever shape or length used in the present invention, the microneedles must be suitable to provide carboxytherapy treatment to a patient in need of such treatment. The microneedles are therefore in fluid communication with a source of CO₂ when the microneedle/base elementelement arrangement is in secure attachment with the treatment device as discussed further herein.

When secured to the base material, microneedles can be oriented substantially perpendicular or at an angle thereto. Still further, the microneedles can be oriented substantially perpendicular to the base material. In some aspects, a configuration of microneedles can comprise an arrangement of comprising different microneedle orientations, heights, or other parameters.

Generally, the microneedles should have the mechanical strength to resist distortion (such as bending) while being inserted into the skin and while being removed one or more times.

The microneedles can be made from medical-grade steel, such as an acupuncture-type needle, and be from about 0.1 to about 0.5 mm in diameter. Each microneedle can be from about 0.4 to about 2.1 mm in length. In aspect of the invention, each microneedle is from about 0.5 mm and 1.1 mm. Still further, the length of each microneedle can be from about 0.1, 0.3, 0.5, 0.7, 1.0, 1.5, 2.0 or 2.5 mm in length, where any one of these lengths can be used individually or in combination in secure attachment to a base material and to be in fluid communication with a source of CO₂ as discussed elsewhere herein.

As noted, microneedles must be securely attached to the base material. In one aspect, the microneedles and a base material to which they are attached comprises a single, disposal one time use unit, otherwise known as a “consumable.” Use of a consumable configuration that securely attaches to a reusable treatment device can facilitate maintenance of a sterile treatment regime, as well as a treatment that is customizable to each patient.

The base material to which the microneedles are securely fastened to provide a microneedle/base element can comprise a rigid material that is sufficiently stiff so as to assist in directing the attached microneedles through a patient's skin. In this regard, the microneedles and base material can be configured as a single unit, such as from stamping of a stainless steel using precision methods that will create needled projections from a flat or substantially flat base material. Use of a metallic material can facilitate application of electrical stimulation and/or heat treatment to a patient in conjunction with carboxytherapy treatment as discussed elsewhere herein. In this regard, the microneedles and base materials are comprised of the same material.

Still further, the base material can comprise flexible material to allow the base material to generally conform to the contours of the skin and to adapt to deformations that may occur when the microneedles are inserted. In this regard, microneedles can be securely attached to the base material such as by embedding them into a polymeric material and then applying a suitable adhesive so as to ensure attachment. A flexible surface can facilitates more consistent penetration during use, since penetration can be limited by deviations in the attachment surface.

The depth of CO₂ infusion will vary according to the treatment, for example, the treatment can be from about 2 to about 20 mm. Microneedle length will be substantially equal to the depth of infusion

### Skin Treatment Device

One aspect of the present application relates to a skin treatment device that is capable of effectively delivering a treatment fluid into the cutaneous and/or subcutaneous tissue of a target body area. A benefit of the skin treatment device of the present application is that multiple insertions of the treatment fluid below the skin surface can be effected simultaneously by a practitioner at a controlled flow rate. In some embodiments, the treatment fluid is a gas or a mixture of gases. In other embodiments, the fluid is a liquid. In other embodiments, the fluid is a gasified liquid. In a particular embodiment, the fluid is CO₂ gas.

In one embodiment, the skin treatment device of the present application comprises a housing having a contact surface. The contact surface comprises a plurality of hypodermic needles for penetrating the skin and delivering the treatment fluid into or under the skin layer. The plurality of needles on the contact surface are in fluid communication with the treatment fluid source to allow delivery of the treatment fluid from the treatment fluid source into or through the skin. In some embodiments, the plurality of needles protrude between about 0.1 mm and about 10.0 mm from the contact surface. In some further embodiments, the plurality of needles protrude between about 0.15 mm and about 4.0 mm from the contact surface. In further embodiments, the plurality of needles protrude between about 0.3 mm and about 3.0 mm from the contact surface. In still further embodiments, the plurality of needles protrude between about 0.3 mm and about 2.0 mm from the contact surface. In some embodiments, the needles have a length of, or about, 0.1 mm, 0.15 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 6.0 mm, 7.0 mm, 8.0 mm, 9.0 mm or 10.0 mm. The needle lengths can control the depth levels and, thus, the exact location of where the treatment fluid will be delivered.

In some embodiments, the needles on the contact surface of the device are all the same length. In other embodiments, the needles on the contact surface of the device are of mixed lengths in order to protrude different depths into or through the body tissue. Needles can be manufactured from stainless steel, and the needle fracture force is hundreds of times greater than the skin insertion force.

In order to cause as little discomfort on the patient as possible during the use of the skin treatment device, the gauge of the needles should be small enough to minimize pain and scarring to the patient. In some embodiments, the size of the needles is sufficient to overcome natural resistance to pierce the stratum corneum. In some embodiments, the needles are between about 26 gauge and about 36 gauge. In some further embodiments, the needles are between about 27 gauge and about 34 gauge. In other further embodiments, the needles are between about 28 gauge and about 33 gauge. In still other further embodiments, the needles are between about 29 gauge and about 32 gauge. In yet further embodiments, the needles are between about 30 gauge and about 32 gauge. In some embodiments, the needles are selected from a gauge of 26, 26s, 27, 28, 29, 30, 31, 32, 33, 34, 35 or 36. In some other embodiments, the needles are microneedles. In some embodiments, the needles on the contact surface of the device are all the same gauge. In other embodiments, the needles on the contact surface of the device are of mixed gauges in order to deliver different volumes of the treatment fluid to different areas of the body tissue.

The size and spacing of the needles may vary depending on the needs of the treatment regimen. In some embodiments, the needles are spaced at about 2, 3, 4, 5, 6, 7, 8, 9, 10 mm apart from each other. The distance between any two needles may be constant or variable. In other embodiments, the needles are arranged in an average density of 1, 2, 3, 4, 5, 6, 7, 8, 9 or 10 needles per cm² of the contact surface. As used herein, the average needle density of the contact surface is defined as the total number of needles divided by the total surface area of the contact surface.

In some embodiments, the contact surface has a total area of about 10-500 cm², about 20-400 cm² or about 40-200 cm². In other embodiments, the contact surface has a total area of about 20 cm², about 40 cm², about 60 cm², about 80 cm², about 100 cm², about 150 cm², about 200 cm², about 250 cm², about 300 cm², about 350 cm², about 400 cm², about 450 cm² or about 500 cm². In some embodiments, the contact surface of the skin treatment device is detachable from the housing and contact surfaces of various sizes and shapes can be attached to the skin treatment device depending on the treatment area and treatment method. In some embodiments, the contact surface has a degree of flexibility that will allow the contact surface to adjust to contours of the body surface to be treated, such as the curvature of the head, thigh etc.

In some embodiments, the contact surface is in the form of a roller. In certain embodiments, the roller has a cylindered shape with a diameter of about 2, 3, 4, 5, 6, 7, 8, 9 or 10 cm and a width (i.e., the distance from side end to side end of the roller) of about 4, 6, 8, 10, 12, 14, 16, 18, 20 cm. In some embodiments, the roller can have a degree of flexibility that will allow the roller to adjust to contours of the body surface to be treated, such as the curvature of the head, thigh etc.

In some embodiments, the housing further comprises a one or more valves that control the flow rate of the treatment fluid. In other embodiments, the housing further comprises a battery. In other embodiment, the house further comprises a control module that controls the fluid flow rate through the one or more valves. In some embodiments, the housing further comprises a fluid inlet to be connected to a fluid source. In other embodiments, the housing comprises a fluid tank located within the housing. In some embodiments, the contact surface is removable and disposable. In other embodiments, the skin treatment device is designed for single use.

FIGS. 1-5 show an embodiment of the skin treatment device 100 designed to introduce CO₂ into a skin tissue of a patient. FIG. 1 depicts the contact surface 110 of a housing 120 (see FIG. 5). In some embodiments, the contact surface 110 is composed of a flexible material such as, but not limited to, polyethylene, polyurethane, silicon or rubber. In other embodiments, the contact surface 110 is composed of a rigid polymer material such as, but not limited to, polystyrene, polycarbonate or polyvinyl chloride. In other embodiments, the contact surface 110 is composed of a rigid polymer material coated with a layer of flexible material. In yet other embodiments, the contact surface 110 is composed of a biodegradable material. The biodegradable material may be selected from any suitable biodegradable material, or from mixtures of two or more thereof. Suitable materials include, but are not limited to, cellulose and cellulosic derivatives, polymers of lactic acid (PLA) and its derivatives, polymers of hydroxyalkanoates (PHAs), biodegradeable copolyesters and polycaprolactones. The biodegradable material may comprise a true biopolymer (PHA or PLA for example), or suitably biodegradable synthetic polymers or suitable mixtures of two or more thereof.

In this non-limiting example, groups of needles are embedded in nodules 112 present on protuberances 114 in the contact surface 110, which is located on the exterior side of a detachable head 122 of the housing 120. Prior to charging the device with the CO₂ gas, the piercing tips of the needles are hidden in the nodules. Because the contact surface 110 is the only part of the skin treatment device 100 that makes physical contact with the body of the patient, in some embodiments, the detachable head 122 of the housing 120 is disposable. In other embodiments, the detachable head 122 of the housing 120 is sterilizable. In some embodiments, the nodules can be used to stimulate, or massage, the skin of the patient prior to injection of the CO₂ gas. When the device is charged with CO₂ gas, the pressure of the CO₂ gas extends the piercing tips of the needles out of the nodules and through the skin of the patient, injecting the CO₂ gas through the skin. In some embodiments, this stimulation or massage of the skin with the nodules serves to mitigate the pain of the needles piercing the skin.

FIG. 2 depicts the interior side 124 of the detachable head 122 of the housing 120. In some embodiments, the interior side 124 of the detachable head 122 comprises chambers through which the CO₂ gas flows to the needles 112 embedded in the contact surface 110. In some embodiments of the skin treatment device 100, the interior side 124 of the detachable head 122 has multiple CO₂ distribution chambers 126 that promotes more even pressure distribution of the CO₂ gas to all of the needles. The CO₂ distribution chambers 126 contains apertures or nodules 125 and are sealed against the base 128 of the housing 120 by rubber seals 127.

FIG. 3 shows the base 128 of the housing 120. In some embodiments, the base 128 comprises valves 129 that contact apertures or nodules 125 in the interior side 124 of the detachable head 122. In some further embodiments, the valves 129 are pin valves that contact apertures/nodules 125. Opening the valves 129 allows the flow of CO₂ gas into the chambers 126 and through the needles 112 embedded in the contact surface 110 into or through the target skin tissue of the patient.

FIG. 4 shows a control module 130 of the skin treatment device 100. In some embodiments, the control module 130 comprises controls or buttons 132 for opening and closing the valves 129, allowing or stopping the flow of CO₂ gas through to the needles 112. The control module 130 may further comprises one or more lights 134. In some embodiments, at least one light indicates the readiness status of the device for application of CO₂. In some embodiments, at least one light indicates the power charge status of the device. In some embodiments, the housing further comprises one or more batteries for powering the device. In some further embodiments, the battery is rechargeable. In alternative further embodiments, the battery is replaceable. In other embodiments, the skin treatment device 100 is powered by a cord that attaches to a separate control device or by an electrical cord to an external power source. The housing 120 further comprises a means for establishing fluid communication of the CO₂ source with the needles that deliver the CO₂ into or through the body tissue. In some embodiments, the CO₂ source is an external tank or reservoir. In alternative embodiments, the CO₂ source is a pressurized CO₂ cartridge that inserts into, or attaches to, the housing 120 of the device. In some embodiments, the CO₂ cartridge is disposable, being replaceable for each new patient. In some embodiments, the controls or buttons 132 or lights 134 are not located on the top of the device, but can be located on the sides, front or back of the device.

FIG. 5 is a side view of an exemplary embodiment of the housing 120. In this non-limiting example, the control module 130 of the housing 120 is knob-shaped to allow easy gripping and manipulation by the gloved hand of a practitioner. In some embodiments, CO₂ gas enters the device from the CO₂ source by way of a tube 140 connected to the device.

In another non-limiting example of the skin treatment device 100, the flat contact surface 110 of the device can be in the form of a roller element. In some embodiments, a plurality of needles are mounted on the roller element, wherein the roller is rotatably mounted on an axle or other suitable configuration of the device that will allow the roller to be rotatable as contemplated herein. The axle or other suitable configuration can include one or a plurality of passageways to allow the CO₂ to be in fluid communication with the needles. Being in fluid communication with a source of CO₂, the plurality of needles suitably allow infusion of CO₂ to a patient in need of such treatment when one or more of the plurality of needles is below the surface of a patient's skin. The number of needle rows that make up the contact surface of the roller element of the present device can vary according to the desired treatment regimen.

In some embodiments, the roller device comprises a plurality of needles permanently attached to the roller device. In some embodiments, the roller element can be disengaged from the device via removable connection. In some embodiments, the roller element is disposable, being replaceable for each new patient. In other embodiments, the roller unit is sterilizable.

In some embodiments, the plurality of needles in the roller element are removable and replaceable. In one embodiment of this removable needle configuration, the needles are integrated into a disposable roller cover. The roller cover can be securely attached to the roller, whereby the roller element is suitably perforated to allow CO₂ to pass into the needles integrated into the roller cover. When the needled roller cover is mounted on the perforated roller element, CO₂ will pass into the skin of the patient when the roller device is placed on a patient's skin. This needled roller cover can enhance safety of the roller device of the present application. When the disposable roller cover is removed from the roller after use, the roller element can be sterilizable to further enhance safety.

In some embodiments, the skin treatment device 100 is attached to a pressured treatment fluid source, such as a CO₂ tank or cartridge. In such embodiments, there is little to no need to include a piston or other type of configuration in order to provide adequate delivery of therapeutic materials under the skin. In some embodiments, the skin treatment device and method of the present application does not comprise a piston or other form of delivery enhancement mechanism. In one embodiment, the present application introduces the treatment fluid into or beneath the skin surface substantially by the pressure from the pressured treatment fluid source.

Another embodiment of a skin treatment device is shown in FIGS. 6-9.

FIG. 6 show one configuration of a treatment device 100 having a handle portion 202. At a first end of handle portion 202, a hose element 204 is attached to a source of CO₂ gas (not shown). As an optional feature, a regulator 206 can be included on treatment device 100. A switch 208 can facilitate operation. In use, end piece 210 is attachable to microneedle/base element 212. Microneedle 214 and base material 216 are also shown.

Referring to FIG. 7, microneedle/base element 212 is shown in contact with patient's skin 300 and in penetration to an interior skin portion 302. The microneedles 214 have a hollow end 218 that will fill with CO₂ gas (not shown) in a penetration area 304.

Referring to FIG. 8, an exemplary configuration of a microneedle/base element 220 is shown. An area of base material 216 is provided with a plurality of microneedles 214. Referring to FIG. 9, another exemplary configuration of a microneedle/base element 222 is shown. An area of base material 216 is provided with a plurality of microneedles 214. The shape of the base element is not limited to the exemplified shapes. The present application envisions any shape of base element that is suitable for carrying out the methods disclosed in the present application. The shape may be determined by the contours of the area of the body to be treated or by the need to avoid a particular bodily feature.

In another embodiment, the device of the present application comprises a plurality of interchangeable head units with rings of microneedles of varying diameters. For example, the device comprises a first interchangeable head with a ring, or rings, of microneedles with a large diameter so that when the ring(s) is/are centered on the skin disorder being treated, the ring(s) is/are at the greatest distance from the skin disorder. The device further comprises one or more interchangeable heads having ring(s) of smaller diameter than the ring(s) on said first interchangeable head, thereby bringing the ring(s) closer to the skin disorder being treated.

### Skin Treatment System

Another aspect of the present application relates to a skin treatment system comprising a self-contained treatment fluid source, a pressure regulator for regulating the pressure of the treatment fluid, an filter for filtering out contaminants such as bacteria, viruses and other gaseous impurities in the treatment fluid, a heating system for raising the temperature of the treatment fluid, a flow regulator for controlling the flow of the treatment fluid, and a skin treatment device for delivering the treatment fluid into or beneath the skin of a patient. In some embodiments, the treatment fluid is CO₂ gas. In a further embodiment, the CO₂ gas is medical grade CO₂ gas, thereby reducing or eliminating the need for a filter.

In an exemplary use of the skin treatment device of the present application, the contact surface or the roller of the skin treatment device is directed over the skin of a patient so that at least some of the plurality of needles projecting from the contact surface penetrate the skin of the patient. During the time that such needles are below the surface of the skin, the treatment fluid, such as CO₂, is applied from the treatment fluid source. Since the needles are in fluid communication with the treatment fluid source₂, the treatment fluid will suitably travel into and below the skin of the patient in a desired amount over a desired period time to provide the desired treatment. Depending the length of the needles, the treatment can be conveyed at various distances below the patient's skin surface. In the case of a roller type contact surface, the frequency of the movement of the roller over the skin determines the number of puncture channels, which can be controlled specifically and thus also the degree to which the treatment fluid can penetrate the patient's skin.

### Kit

In one aspect, a treatment device of the present application, the microneedle/base element and one or more companion products are packaged together and marketed as a kit. The examples of the items in the kit may include, but are not limited to, the device including a pre-determined number of disposable or replaceable microneedle/base element configurations, a reusable treatment device for application of carboxytherapy and one or more additional treatments of electrical stimulation, galvanic action or heat therapy, a topical treatment composition in a suitable container/dispenser (such as a tube, a bottle, a pump, a jar, a dropper, a or unit-dose dispenser) to be used before, during, or after the device application. Additionally, the kit may also contain a cleansing product to be used to sanitize/sterilize the skin prior to the device application. The kit may also include a film forming composition or bandage to be used after treatment to protect the treated skin site and to enhance the therapeutic efficacies for the treated skin.

### Methods of Treatment

Treatment in accordance with the methods of the present application may be localized, such that the target site of a pimple or other blemish, a wrinkle, a razor bumps/ingrown hairs, a herpes sore, a skin infection, an age-spot, or any other skin disorder. Still further, the treatment can be used on larger areas such as the scalp (to enhance hair growth) or the thighs or other areas (for example, to treat cellulite).

In one aspect, a patient is treated by: (a) affixing a treatment device comprising a plurality of microneedles securely attached to a surface material; (b) applying pressure to the device such that one or more of the plurality of microneedles penetrates the skin of the patient; (c) applying a carboxytherapy treatment; and optionally one or more of a galvanic treatment, electrical stimulation or heat treatment; and (d) removing the microneedles from the patient's skin.

In an exemplary aspect, a medical professional places a treatment device so that the microneedle/base element is in contact or substantially in contact with a location of the skin of a patient. The treatment device is activated by the user, such as by using a plunger, piston or the like, so that at least some of the plurality of microneedles projecting from the base material penetrates the skin of the patient. During the time that such needles are below the surface of the skin, CO₂ is applied from a source and, since the needles are in fluid communication with the source of CO₂, the gas will suitably travel into and below the skin of the patient thereby providing a carboxytherapy treatment. Being in fluid communication with a source of CO₂, the plurality of needles suitably allow application of carboxytherapy to a patient in need of such treatment when one or more of the plurality of microneedles is below the surface of a patient's skin.

By way of example, application of carboxytherapy for intradermal treatments such as skin tightening, rejuvenation, stretch marks and hair regrowth, flow rates for CO₂ gas can be from about 5 to about 300 ml/min. In some embodiments, the CO₂ flow rates are from 50 to about 200 ml/minute, or from about 80 to about 120 ml/min. Still further, CO₂ flow rates can be from about 5, 10, 15, 20, 30, 40, 50, 60, 70, 80, 90, 100, 110, 120, 150, 170 or 200 ml/min, where any value can form an upper or lower endpoint, as appropriate. In some embodiments, the flow rates for CO₂ gas are in the range of 5-10 ml/min, 5-20 ml/min, 5-40 ml/min, 5-80 ml/min, 5-100 ml/min, 5-120 ml/min, 5-150 ml/min, 5-200 ml/min, 5-250 ml/min, 10-20 ml/min, 10-40 ml/min, 10-80 ml/min, 10-100 ml/min, 10-120 ml/min, 10-150 ml/min, 10-200 ml/min, 10-250 ml/min, 10-300 ml/min, 15-20 ml/min, 15-40 ml/min, 15-80 ml/min, 15-100 ml/min, 15-120 ml/min, 15-150 ml/min, 15-200 ml/min, 15-250 ml/min, 15-300 ml/min, 20-40 ml/min, 20-80 ml/min, 20-100 ml/min, 10-120 ml/min, 20-150 ml/min, 20-200 ml/min, 20-250 ml/min, 20-300 ml/min, 40-80 ml/min, 40-100 ml/min, 40-120 ml/min, 40-150 ml/min, 40-200 ml/min, 40-250 ml/min, 40-300 ml/min, 80-100 ml/min, 80-120 ml/min, 80-150 ml/min, 80-200 ml/min, 80-250 ml/min, 80-300 ml/min, 100-120 ml/min, 100-150 ml/min, 100-200 ml/min, 100-250 ml/min, 100-300 ml/min, 120-150 ml/min, 120-200 ml/min, 120-250 ml/min, 120-300 ml/min, 150-200 ml/min, 150-250 ml/min, 150-300 ml/min, 200-250 ml/min, 200-300 ml/min, or 250-300 ml/min. In other embodiments, the CO₂ gas is supplied at a variable flow rate within a single treatment or among multiple sessions of treatments. In some embodiments, a single treatment session comprises a period of high flow rate (e.g., 100-200 ml/min), a period of medium flow rate (e.g., 40-99 ml/min), and a period of low flow rate (e.g., 10-39 ml/min). In some embodiments, a complete treatment regimen comprises one or more sessions at a high flow rate (e.g., 100-200 ml/min), one or more sessions at a medium flow rate (e.g., 40-99 ml/min), and one or more sessions at a low flow rate (e.g., 10-39 ml/min). Time for such treatments can vary from about 30 seconds to about 180 seconds or from about 45 seconds to about 90 seconds. Time for treatment is from about 30, 45, 60, 75, 90, 105, 120, 135, 150, 165 or 180 seconds, where any value can form an upper or lower endpoint, as appropriate.

By way of further example, for subcutaneous injections for fat reduction in areas such as the chin, arms, knees, thighs, stomach or buttocks, flow rates for the CO₂ gas can be from about 40 to about 360 ml/min or from about 90 to about 240 ml/min. Still further, CO₂ flow rates for subcutaneous carboxytherapy treatment can be from about 5, 10, 15, 20, 30, 40, 60, 80, 100, 120, 140, 160, 180, 200, 220, 240, 260, 280, 300, 320, 340 or 360 ml/min, where any value can form an upper or lower endpoint, as appropriate. In some embodiments, the flow rates for CO₂ gas are in the range of 5-10 ml/min, 5-20 ml/min, 5-40 ml/min, 5-80 ml/min, 5-100 ml/min, 5-120 ml/min, 5-150 ml/min, 5-200 ml/min, 5-250 ml/min, 5-300 ml/min, 5-360 ml/min, 10-20 ml/min, 10-40 ml/min, 10-80 ml/min, 10-100 ml/min, 10-120 ml/min, 10-150 ml/min, 10-200 ml/min, 10-250 ml/min, 10-300 ml/min, 10-360 ml/min, 15-20 ml/min, 15-40 ml/min, 15-80 ml/min, 15-100 ml/min, 15-120 ml/min, 15-150 ml/min, 15-200 ml/min, 15-250 ml/min, 15-300 ml/min, 15-360 ml/min, 20-40 ml/min, 20-80 ml/min, 20-100 ml/min, 20-120 ml/min, 20-150 ml/min, 20-200 ml/min, 20-250 ml/min, 20-300 ml/min, 20-360 ml/min, 40-80 ml/min, 40-100 ml/min, 40-120 ml/min, 40-150 ml/min, 40-200 ml/min, 40-250 ml/min, 40-300 ml/min, 40-360 ml/min, 80-100 ml/min, 80-120 ml/min, 80-150 ml/min, 80-200 ml/min, 80-250 ml/min, 80-300 ml/min, 80-360 ml/min, 100-120 ml/min, 100-150 ml/min, 100-200 ml/min, 100-250 ml/min, 100-300 ml/min, 100-360 ml/min, 120-150 ml/min, 120-200 ml/min, 120-250 ml/min, 120-300 ml/min, 120-360 ml/min, 150-200 ml/min, 150-250 ml/min, 150-300 ml/min, 150-360 ml/min, 200-250 ml/min, 200-300 ml/min, 200-360 ml/min, 250-300 ml/min, 250-360 ml/min, or 300-360 ml/min. In some embodiments, a single treatment session comprises a period of high flow rate (e.g., 200-360 ml/min), a period of medium flow rate (e.g., 50-199 ml/min), and a period of low flow rate (e.g., 5-49 ml/min). In some embodiments, a complete treatment regimen comprises one or more sessions at a high flow rate (e.g., 200-360 ml/min), one or more sessions at a medium flow rate (e.g., 50-199 ml/min), and one or more sessions at a low flow rate (e.g., 5-49 ml/min). Treatment times for subcutaneous fact treatment can be from about 1 to about 8 minutes, or from about 2 to about 6 minutes. Still further, the treatment time for subcutaneous injection of CO₂ can be from about 1, 1.5, 2, 3, 3.5, 4, 4.5, 5, 5.5, 6, 6.5, 7.0, 7.5, 8, 10, 15, 20 or 30 minutes, where any value can comprise an upper or lower endpoint as appropriate.

In one aspect, the microneedle/base element and the treatment device can be configured to allow electric current travel into the skin of the patient during the treatment of a patient. The electricity supplied to the disrupted area may also accelerate healing and other benefits that can magnify the therapeutic benefits of carboxytherapy alone. Alternatively, the microneedles can comprise a conductive material so as to allow application of heat (and/or to heat the CO₂) during a treatment.

In one aspect of the present invention, prior to activation of electrical stimulation in the device, the microneedles are inserted into the skin of the patient to disrupt the skin at the desired location(s) thereby, increasing the electric current passage at the selected skin locations to enhance the desirable effect of electric stimulation.

The electrical aspect of the treatment device of the present invention can be powered by powered by a power source, such as battery, piezoelectric, electric-mechanical (e.g., a coil magnet), or by a galvanic couple, the disclosure of which is incorporated in its entirety by this reference, so that processes of stratum corneum disruption and electric stimulation are conducted with the same device without the need of changing devices during the treatment.

Still further, the skin treatment device of the present invention can comprise suitable materials to provide galvanic action. In this aspect, the microneedles and/or the base material can be made from two dissimilar metals in contact with each other so that they form a galvanic couple, and are therefore capable of generating a galvanic current when the microneedles/base material contacts an electrolyte-containing medium. For example, the base material can comprise a thin zinc sheet, fabricated with the manufacture methods disclosed in U.S. Pat. Nos. 5,983,136, 6,532,386, 6,050,988, or 6,219,574, (which disclosures are incorporated herein in their entireties by this reference) while another metal (e.g., silver, silver-silver chloride, copper, gold) can be coated on all or part of the areas of one or more of the plurality of microneedles.

During a skin treatment, for example, both metals of the galvanic couple (i.e., zinc and silver-silver chloride) on the microneedle/base element member can be in contact with an electrolyte medium (e.g., a topical composition, or a body fluid such as sweat) and/or the skin to act as a galvanic cell (e.g., of approximately 1 volt) and to generate an electric current, going out from the zinc positive electrode, passing through the electrolyte medium and/or the skin, and returning into the silver-silver chloride negative electrode.

Alternatively, the two metals forming the galvanic couple may be made to contact the third metal (e.g., titanium, or stainless steel) from which can be configured on or within one or more of the microneedle material or base element material. For example, a zinc layer may be coated onto the selective areas of a titanium or stainless steel microneedles member by electric plating, electroless plating, or using a conductive ink including a zinc powder and a polymer binder. Similarly, a silver-silver chloride layer may be coated to other areas of a titanium or stainless steel microneedles. The conductive metallic microneedle serves as a lead to connect the galvanic elements zinc and silver-silver chloride. A galvanic current is generated when both galvanic elements coming into contact with the electrolyte medium and/or the skin during the device application.

In addition, in order to further enhance electrical stimulation and or galvanic action efficacy, the skin of the patient can first be treated with a relatively high concentration of cosmetically acceptable organic solvent, (e.g., glycerin, propylene glycol, or polyethylene glycol), or a non-conductive solute (e.g., low molecular weight sugars, dextrans, or urea).

Another aspect of the present application relates to a method of applying carboxytherapy to a subject in need of such treatment. The method comprises the steps of: contacting a target body surface of a subject with a treatment device comprising: a plurality of hollow needles attached to a contact surface of a housing; and a CO₂ source in fluid communication with at least one of the plurality of hollow needles; applying pressure to the housing such that one or more of the plurality of hollow needles penetrate an epidermis or an outermost layer of cell in the target body surface; skin of the patient; applying a therapeutic amount of CO₂ to the subject through the plurality of hollow needles; and removing the plurality of hollow needles from the target body surface.

In some embodiments, the subject is a mammal. In other embodiments, the mammal is a human. In other embodiments, the mammal is a domestic animal, such as a dog, a cat, a monkey, a rat, a mouse, a rabbit, a guinea pig and the like. In other embodiments, the mammal is a farm animal, such as a cow, a horse, a pig, a sheep, a goat, and the like. In yet other embodiments, the mammal is a zoo animal.

In some embodiments, the method further comprises the step of subjecting the target body surface to one or more additional treatments selected from the group consisting of galvanic treatments, electrical stimulations, heat treatments and light treatments. In some other embodiments, the one or more additional treatments are provided concurrently with the carboxytherapy using the same treatment device. In some other embodiments, the one or more additional treatments are provided prior to or after the carboxytherapy. In some other embodiments, the method further comprises the step of applying to the target body surface an effective amount of a treatment composition formulized for topical administration.

In some embodiments, the plurality of hollow needles are microneedles having a diameter of about 0.1 mm to about 0.5 mm and a length of about 0.4 mm to about 2.1 mm.

In some embodiments, the target body surface is suffering from a skin condition selected from the group consisting of acne, psoriasis, skin infections, blemishes, hyperpigmentation, hypopigmentation, alopecia, excessive hair growth, unwanted hair growth, rough skin, dry skin, lax skin, wrinkles, hypervasculatated skin, sebum production disorders, excessive pore appearance, excessive perspiration, hyperhidrosis, tattoo appearance, rashes, scar appearance, pain, itch, burn, inflammation, warts, corns, calluses, edema, ivy/oak poisoning, and bites from insects, spiders, snake, and other animals. In one embodiment, the target body surface is scalp suffering from hair loss. In another embodiment,

the target body surface is suffering from alopecia areata. In another embodiment, the target body surface is suffering from diabetic ulcer. In another embodiment, the target body surface is suffering from straie. In yet another embodiment, the target body surface is a scarred body surface.

Another aspect of the present application relates to a method for treating hair loss in a target area, such as scalp. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue.

In some embodiments, the CO₂ is introduced into the target area using the skin treatment device and/or the of the skin treatment system of the present application. The plurality of needles in the skin treatment device are selected to have a size and spaced relationship suitable for hair loss treatment.

In some embodiments, the CO₂ is introduced at a flow rate of 50-200 ml/min and for a period of 10-60 minutes.

In other embodiments, the introducing step is repeated 4-20 times with an interval of about 24-72 hours between any two repeats.

In some embodiments, the method further includes the step of applying a local anesthetic to the target area prior to the introducing step. Examples of local anesthetic include, but are not limited to, of procaine, amethocaine, cocaine, lidocaine, prilocalne, bupivacaine, levobupivacaine, ropivacaine, mepivacaine and dibucaine, etidocaine, chloroprocaine, sarapin, benzocaine, tetracaine, pramoxine, oxyprocaine, dyclonine, propoxycaine, chloroxylenol, cinchocaine, dexivacaine, diamocaine, hexylcaine, pyrrocaine, risocaine and rodocaine.

In some embodiments, the method further include the step of applying a composition comprising a hair growth promoting agent to the target area, wherein the composition is formulated for topical application. Examples of the hair growth promoting agents include, but are not limited to, hair growth factors, minoxidil, finasteride and kopexil, including analogues and derivatives therefrom; cyclosporin 7-thioamide, donepezil hydrochloride, antiandrogenic agents, bimatoprost, Sophora flavescens extract, Serenoa serrulata fruit extract, Serenoa repens extract, licorice extract.

Another aspect of the present application relates to a method for treating a skin condition in a target area. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue. In some further embodiments, the subcutaneous layer comprises adipose tissue

In some embodiments, the CO₂ is introduced into the target area using the skin treatment device and/or the of the skin treatment system of the present application. The plurality of needles in the skin treatment device are selected to have a size and spaced relationship suitable for the treatment.

In some embodiments, the CO₂ is introduced at a flow rate of 40-360 ml/min and for a period of 2-120 minutes.

In other embodiments, the introducing step is repeated 1-40 times with an interval of about 6-120 hours between any two repeats.

In some embodiments, the method further includes the step of applying a local anesthetic to the target area prior to the introducing step. Examples of local anesthetic include, but are not limited to, of procaine, amethocaine, cocaine, lidocaine, prilocalne, bupivacaine, levobupivacaine, ropivacaine, mepivacaine and dibucaine.

Examples of skin conditions include, but not limited to, eczema, seborrhea, vitiligo, lentigo, scleroderma, sunburn, sun damaged skin, estrogen imbalance, hyperpigmentation, hypopigmentation, wrinkles and coarse deep wrinkles, fine lines, skin lines, undereye circles, crevices, bumps, large pores, unevenness, surface roughness, blotchiness, sallowness, loss of skin elasticity, discoloration, yellowing, age spots, freckles, skin atrophy, skin breakout, skin fragility, dryness, chapping, sagginess, thinning, hyperplasia, hyperkeratinization, elastosis, fibrosis, enlarged pores, cellulite formation, bruising, acne vulgaris, cystic acne, acne scars, keloid scars, hypertrophic scars, striae (e.g., stretch marks), dermatitis (e.g., seborrheic dermatitis, nummular dermatitis, contact dermatitis, atopic dermatitis, exfoliative dermatitis, perioral dermatitis, and stasis dermatitis), dermal and epidermal hypoplasia, folliculitis, enlarged pores, ichthyoses (e.g., ichthyosis hystrix, epidermolytic hyperkeratosis, and lamellar ichthyosis), follicular disorders (e.g., pseudofolliculites, senile comedones, nevus comidonicas, and trichostatis spinulosa), benign epithelial tumors (e.g., flat warts, trichoepithelioma, and molluscum contagiosum), perforated dematoses (e.g., elastosis perforans seripiginosa and Kyrles disease), disorders of keratinization (e.g., Dariers disease, keratoderma, hyperkeratosis plantaris, pityriasis rubra pilaris, lichen planus acanthosis nigricans, and psoriasis), pityriasis (e.g., pitiyriasis rosea, pityriasis rosacea and pityriasis rubra), keratoses, impetigo, erysipelas, erythrasma, eczema, senile purpura, excessive sebum (oil) production, sebaceous hyperplasia (enlarged oil glands), viral infections, fungal infections, bacterial infections, spider veins (telangectasia), hirsutism, rosacea, pruritis, calluses, warts and corns.

In some embodiments, the method further include the step of applying a composition comprising a therapeutic agent to the target area, wherein the composition is formulated for topical application. Examples of the therapeutic agents include, but are not limited to, antimicrobial agents, analgesic and/or non-steroidal anti-inflammatory (NSAID) agents, steroidal/corticosteroidal agents, wound repair agents, anti-cancer agents and skin benefit agents. Exemplary antimicrobial agents include, but are not limited to, anti-bacterial agents (e.g., clindamycin and erythromycin, zithromycin, minocycline, tetracycline, kanamycin, metronidazole, neomycin, bacitracin, polymixin, mafenide acetate, silver sulfadiazine, gentamicin sulfate; anti-fungal agents (terbinafine, itraconazole, micronazole nitrate, thiapendazole, tolnaftate, clotrimazole and griseofulvin caprylyl glycol, triclosan, phenoxyethanol, nystatin or clortrimazole); anti-viral agents (e.g., acyclovir, brivudine, cidofovir, desciclovir, didanosine, famciclovir, 5-fluorouracil, 2-deoxy- and 5-deoxy-5-fluorouridine, ganciclovir, idoxuridine, lamivudine, lobucavir, penciclovir, retrovir, sorivudine, trifluridine, valacyclovir, valganciclovir, vidarabine, zalcitabine, zidovudine, imiquiod, docosanol, brivudin, interferon, famvir, cidofovir, podophyllin, podophyllotoxin); analgesic agents (e.g., aspirin, nonsteroidal anti-inflammatory agents (NSAIDs), salisylates, including salicylic acid, methyl salicylate, olsalazine, sulfasalazine and salsalate; diflunisal, para-aminophenol derivatives, acetanilide, acetaminophen, phenacetin, fenamates, mefenamic acid, meclofenamate, sodium meclofenamate, heteroaryl acetic acid derivatives, tolmetin, ketorolac, diclofenac, propionic acid derivatives, ibuprofen, naproxen sodium, naproxen, fenoprofen, ketoprofen, flurbiprofen, oxaprozin; enolic acids, oxicam derivatives, piroxicam, meloxicam, tenoxicam, ampiroxicam, droxicam, pivoxicam, pyrazolon derivatives, phenylbutazone, oxyphenbutazone, antipyrine, aminopyrine, dipyrone, coxibs, celecoxib, rofecoxib, nabumetone, apazone, indomethacin, sulindac, etodolac, isobutylphenyl propionic acid, lumiracoxib, etoricoxib, parecoxib, valdecoxib, tiracoxib, etodolac, darbufelone, dexketoprofen, aceclofenac, licofelone, bromfenac, loxoprofen, pranoprofen, piroxicam, nimesulide, cizolirine, 3-formylamino-7-methylsulfonylamino-6-phenoxy-4H-1-benzopyran-4-one, meloxicam, lornoxicam, d-indobufen, mofezolac, amtolmetin, pranoprofen, tolfenamic acid, flurbiprofen, suprofen, oxaprozin, zaltoprofen, alminoprofen and tiaprofenic acid); opioid analgesics (e.g., propoxyphene, meperidine, hydromorphone, dihydromorphine, pethidine, hydrocodone, oxycodone, morphine, codeine, and tramodol); NMDA antagonist analgesics (e.g., 2-piperidino-1 alkanol derivatives, ketamine, dextormethorphan, eliprodil, and ifenprodil); skeletal muscle relaxant analgesics (e.g., flexeril, carisoprodol, robaxisal, norgesic, and dantrium); cell differentiating and anti-proliferative agents (e.g., retinoids, such as retinol, retinal, and retinyl esters, such as retinyl acetate, retinyl butyrate, retinyl propionate, retinyl octanoate, retinyl laurate, retinyl palmitate, retinyl oleate, and retinyl linoleate), vitamin D, or vitamin D analogs (calcipotriene)); chemodenervation agents include botulinum type A and/or type B toxins, as well as the serotype C-G toxins; mitochondrial inhibitors (e.g., anthraline (dithranol, chrysarobin, or coal tar)); topical steroids (e.g., clobetasol propionate, betamethasone, betamethasone dipropionate, halobetasol propionate, fluocinonide, diflorasone diacetate, mometasone furoate, halcinonide, desoximetasone, fluticasone propionate, flurandrenolide, triamcinolone acetonide, fluocinolone acetonide, hydrocortisone, hydrocortisone valerate, prednicarbate, desonide, or alclometasone dipropionate); immunosuppressive compounds (e.g., tacrolimus (FK-506)); JAK2 inhibitors (e.g., INCB18424); JAK3 inhibitors (e.g., CP-690,550); parathyroid hormone-related protein (PTHrP) agonists (e.g., PTH(1-34)); and cell adhesion blockers (e.g., pan-selectin antagonist bimosiamose), DNA repair enzymes (e.g., bacteriophage T4 pyrimidine dimer-specific endonuclease, Micrococcus luteus N-glycosylase/AP lyase, Saccharomyces cerevisiae N-glycosylase/apurinic-apyrimidinic lyase, Schizosaccharomyces pombe UV damage endonuclease (UVDE), Chlorella virus isolate PBCV-1 pyrimidine dimer-specific glycosylase, and Anacystis nidulans photolyase); methylxanthines for use in the present invention include caffeine (1,3,7-trimethylxanthine), theophylline (1,3-dimethylxanthine), aminophylline, theobromine (3,7-dimethylxanthine), paraxanthine, isobutylmethyl xanthine, butymethylxanthine; salicylic acid, benzoyl peroxide, caffeine, caffeic acid compounds, avobenzone, oxybenzone, octylmethoxycinnamate, titanium dioxide, zinc oxide, dihydroxyacetone, palmitoyl pentapeptide, argireline, interleukins such as IL6 and IL10, growth factors such as EGF and TGF.

Another aspect of the present application relates to a method for promoting wound healing in a target area, such as scalp. The method comprises the step of introducing an effective amount of CO₂ into the subcutaneous tissue of the target area for a sufficient period of time. The effective amount of carbon dioxide is introduced into the target area with a plurality of hollow needles that puncture epidermis of the target area and release carbon dioxide at a desired rate within the subcutaneous tissue.

In some embodiments, the CO₂ is introduced into the target area using the skin treatment device and/or the of the skin treatment system of the present application. The plurality of needles in the skin treatment device are selected to have a size and spaced relationship suitable for the treatment.

In some embodiments, the CO₂ is introduced at a flow rate of 5-300 ml/min and for a period of 10-60 minutes.

In other embodiments, the introducing step is repeated 5-20 times with an interval of about 24-120 hours between any two repeats, or sessions.

In some embodiments, a session of the method of treating a skin disorder comprises several application steps. A first application step comprises the insertion of needles in a ring a great distance from the skin disorder being treated, for example a distance of between two to four inches. For said first application step, the flow rate of the device is relatively high, for example between 120 and 200 ml/min. Said first application step is followed by at least one intermediate step, wherein the ring of insertion is brought closer to the skin disorder being treated than the first application step or the preceding intermediate step. For each intermediate step, the flow rate is reduced from the application step immediately prior, for example to a flow rate of between 40 and 120 ml/min. The session comprises a final application step to the skin immediately around the periphery of (such as for an open wound) or within (such as for scars or stria) the skin disorder being treated. Said final application step is carried out at a relatively low flow rate, for example at a flow rate of between 5 and 20 ml/min.

In some embodiments, the method further includes the step of applying a local anesthetic to the target area prior to the introducing step. Examples of local anesthetic include, but are not limited to, of procaine, amethocaine, cocaine, lidocaine, prilocalne, bupivacaine, levobupivacaine, ropivacaine, mepivacaine and dibucaine.

In some embodiments, the method further include the step of applying a composition comprising wound healing promoting agent to the target area, wherein the composition is formulated for topical application. Examples of the wound healing promoting agents include, but are not limited to, TGF-related growth factors (TGF-β1, TGF-β2, TGF-133), PDGF-related growth factors (PDGF-M, PDGF-BB, VEGF), FGF-related growth factors (a-FGF, b-FGF, KGF), IGF-related growth factors (IGF-1, IGF-II, insulin), EGF-related growth factors (EGF, HB-EGF, TGFα, amphiregulin, betacellulin), HGF/SF, VEGF, CTGF, TNFα, IL-1, IL-2, IL-6, IL-8, γ-interferon, IL-4, IL-10, matrix metalloproteinases (MMPs; MMP-1, -2, -3, -7, -8, -9, 10, -11, -12, -13, -14, -15, -16, -17, -19, -20, -21, -23A, 23B, -24, -25, -26, -27, -28), tissue inhibitors of metallopeptidases (TIMPs), including TIMP-1, -2, -3 and -4, plasmin, serratiopeptidase (Serratia E-15 protease, also known as serralysin, serratiapeptase, serratia peptidase, serratio peptidase, or serrapeptidase). carboxylates, cipemastat, doxycycline, hydroxamates, marimastat, phosphinyls, tetracyclines, thiols and combinations thereof.

The compositions formulated for topical administration may be in the form of a cream, lotion, gel, serum, tonic, emulsion, paste, or spray for topical administration. As used herein, the term “cream” refers to a spreadable composition, typically formulated for application to the skin. Creams typically contain an oil and/or fatty acid based-matrix.

The effective amount of CO₂ depends on the method of treatment. In some embodiments, the effective amount of the CO₂ is defined by the rate of the CO₂ given in the target area and the duration of treatment is a single session. While the effective amount of carbon dioxide may vary from patient to patient, in some embodiments, the CO₂ is introduced into the target area at a flow rate of about 0.1-20 ml/cm²/min, about 0.1-1 ml/cm²/min, about 0.1-2 ml/cm²/min, about 0.1-5 ml/cm²/min, about 0.1-10 ml/cm²/min, about 0.1-15 ml/cm²/min, about 0.5-2 ml/cm²/min, about 0.5-5 ml/cm²/min, about 0.5-10 ml/cm²/min, about 0.5-15 ml/cm²/min, about 0.5-20 ml/cm²/min, about 1-2 ml/cm²/min, about 1-5 ml/cm²/min, about 1-10 ml/cm²/min, about 1-15 ml/cm²/min, about 1-20 ml/cm²/min, about 2-5 ml/cm²/min, about 2-10 ml/cm²/min, about 2-15 ml/cm²/min, about 2-20 ml/cm²/min, about 5-10 ml/cm²/min, about 5-15 ml/cm²/min, about 5-20 ml/cm²/min, about 10-15 ml/cm²/min, about 10-20 ml/cm²/min or about 15-20 ml/cm²/min for a period of about 0.5-60 min, about 0.5-5 min, about 0.5-10 min, about 0.5-20 min, about 0.5-30 min, about 0.5-40 min, about 0.5-50 min, about 2-5 min, about 2-10 min, about 2-20 min, about 2-30 min, about 2-40 min, about 2-50 min, about 2-60 min, about 5-10 min, about 5-20 min, about 5-30 min, about 5-40 min, about 5-50 min, about 5-60 min, about 10-20 min, about 10-30 min, about 10-40 min, about 10-50 min, about 10-60 min, about 20-30 min, about 20-40 min, about 20-50 min, about 20-60 min, about 30-40 min, about 30-50 min, about 30-60 min, about 40-50 min, about 40-60 min or about 50-60 min in a single session.

In some embodiments, the treatment comprises 2-40, 2-30, 2-20, 2-10, 2-5, 4-40, 4-30, 4-20, 4-10, 6-40, 6-30, 6-20, 6-10, 8-40, 8-30, 8-20, 10-40, 10-30, 10-20, 15-40, 15-30, 15-20, 20-40 or 20-30 sessions with an interval of about 12-72, 12-48, 12-24 hours between each two sessions.

The multi-needle approach decreases the time needed to provide a beneficial amount of CO₂ to a patient in need of such treatment without also reducing the efficacy of such a treatment. Moreover, beneficial CO₂ treatment can be applied to large surfaces in a short period time.

In some embodiments, the method of treatment using the device of the present application does not involve the application of any active ingredient other than CO₂. In further aspects, the method of using the device consists essentially of the application of CO₂ below the skin of a patient needing treatment thereof.

The present invention is further illustrated by the following examples which should not be construed as limiting. The contents of all references, patents and published patent applications cited throughout this application, as well as the Figures and Tables, are incorporated herein by reference.

### EXAMPLES

### Example 1

### Treatment of Alopecia Areata

A female patient presented with Alopecia Areata and had been suffering with this condition for over seven years. The patient had been to 14 different specialists in search of an adequate treatment, but was relegated to wearing a wig. No treatments, including the topical application of Minoxidil and hair growth factors had produced satisfactory relief from the condition. The patient was subjected to once-weekly carboxytherapy treatment administered in a clinical setting. Warmed CO₂ was administered at a flow rate of 80 ml/min. Additionally, the patient was instructed to continue once daily topical application of Minoxidil and hair growth factor.

After ten clinical sessions in two and a half months, the patient exhibited significant hair ingrowth into the bald patches. The clinical treatment protocol was reduced to carboxytherapy treatment two times per month. Following 20 sessions of clinical carboxytherapy treatment in about six months, the patient exhibited hair growth throughout the bald patches.

### Example 2

### Treatment of Male-Pattern Baldness

An adult male patient presented with hair loss around the whorl. The patient was clinically treated with carboxytherapy and within three months significant regrowth of hair around the whorl was seen. After 5 months of carboxytherapy, the patient experienced full ingrowth of normal-textured hair around the whorl.

### Example 3

### Treatment of Generalized Hair Thinning

A male patient presented with generalized thinning of the hair across the entire crown of his scalp. The patient was subjected to once-monthly clinical carboxytherapy treatment. At three months, the patient exhibited substantial in-filling of the thinned region. The treatment protocol was changed to twice-monthly administration of carboxytherapy and at five months the patient showed significant coverage of the crow region with normal-textured hair.

### Example 4

### Treatment of Diabetic Ulcers

Ten insulin-dependent diabetic patients with chronic lower extremity wounds were referred for CO₂ transdermal treatment. The control group consisted of five of the patients, three who were claustrophobic and two who refused CO₂ insufflation treatments because of logistic reasons. Five patients underwent 30 CO₂ insufflation treatments in the problem wound protocol (30 min/day, 5 days/wk). All patients were evaluated with transcutaneous oxygen measurements and had an initial surgical debridement of the wound.

An exemplary protocol for a CO₂ insufflation treatment of an ulcerated wound comprises several steps of CO₂ application. A first application step comprises inserting microneedles in a ring of large radius around the wound, for example about two inches from the edges of the wound. CO₂ gas is insufflated at a high flow rate, for example between 120 and 200 ml/min, more particularly about 150 ml/min. This is followed by a second application in a smaller ring, about one inch from the edges of the wound. CO₂ gas is insufflated at a lower flow rate, for example between 50 and 100 ml/min, more particularly about 80 ml/min. This is followed by a third application in a still smaller ring, about one/half inch from the edges of the wound. CO₂ gas is insufflated at a still lower flow rate, for example between 30 and 50 ml/min, more particularly about 40 ml/min. A last application step is made in the skin immediately around the periphery of the wound using a single microneedle applicator using a very low flow rate of between about 5 and 25 ml/min, more particularly about 15-20 ml/min.

Weekly tracings of the wound surface area were made by a nurse or resident who was blinded to the group assignment. At the end of 7 wk, the mean wound area expressed as a percentage of pretreatment baseline area was compared between groups (analysis of variance, Duncan's post hoc). No significant differences were noted between groups with respect to age, gender, baseline wound area, wound site O₂ tension, or presence of osteomyelitis. At the completion of each of the 7-wk treatment periods, a significantly greater reduction in wound surface area was noted in the CO₂ vs. the control group (P<0.05).

A 65 year old male diabetic patient with hypertension and occlusive arterial disease in the lower limbs (ankle-brachial index 0.4) presented with a three-month post-traumatic wound in the metatarsal area of the left foot. Prior to carboxytherapy, the patient was treated with antibiotics and extensive necrotic exeresis with no revascularization procedure. after six applications of carboxytherapy treatment, the wound has begun to close and new skin is forming over the wound after 12 applications of carboxytherapy. Following 16 applications od carboxytherapy treatment, there was a substantial reduction in size of the wound, with the remainder being covered over by a scab. The wound was fully covered over by new skin growth after 20 carboxytherapy application.

A 51 year old male diabetic patient having chronic venous insufficiency in his left lower limb with secondary deep venous thrombosis. The patient presented with open ulcers (clinical, aetiological, anatomical and pathological elements (CEAP) classification C6) that had failed to respond to conventional curative and compressive therapies for the previous 12 months. The patient was started on a course of carboxytherapy treatment, along with maintenance of the conventional therapies already in use. The wound begun to close after four applications of carboxytherapy treatment, and was covered by a scab after eight carboxytherapy applications. There was a reduction in size of the wound after 12 cart treatments and the wound was substantially closed after 16 carboxytherapy treatments.

### Example 5

### Treatment of Straie

Striae, commonly known as stretch marks, can appear when there is rapid stretching of the skin. They are often associated with the abdominal enlargement of pregnancy. They also can be found in children who have become rapidly obese or may occur during the rapid growth of puberty in males and females. Striae are most commonly located on the breasts, hips, thighs, buttocks, abdomen, and flank.

A female patient presented with straie on the buttocks and was treated with a course of once/monthly carboxytherapy treatments at an administration rate of 80 ml/min. Following six carboxytherapy treatments, the appearance of the straie was significantly diminished.

### Example 6

### Treatment of Surgical Scars

Scars are a natural result of some kind of injury to the skin. They can occur because of surgery, accidental injury, acne or infection. As skin heals, the area can become thickened, raised and discolored, resulting in a permanent scar. Some scars fade with time, but most remain visible. Even those that fade may take years to do so. In some instances, scar tissue may cause physical discomfort. In others, visible scars may cause embarrassment or emotional discomfort for a patient, for example, such as scars left after some types of reconstructive surgery.

A patient presented with scars remaining near the ear following a facelift procedure. Following 4 treatments the visibility of the scars was greatly diminished.

A female patient presented with raised, darkened, keloid scars on the breasts and abdomen following reconstructive surgery. The patient was treated with a course of once/monthly carboxytherapy treatment at a flow rate of 80 ml/hr. Following six treatments, the scars were no longer raised and had lightened to more closely approximate the patient's natural skin tone.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A method of applying carboxytherapy to a subject in need of such treatment, comprising the steps of:
contacting a target body surface of said subject with a treatment device comprising:
a plurality of hollow needles attached to a contact surface of a housing; and
a CO₂ source in fluid communication with at least one of said plurality of hollow needles;
applying pressure to said housing such that one or more of the plurality of hollow needles penetrate an epidermis or an outermost layer of cell in said target body surface;
applying a therapeutic amount of CO₂ to said subject through said plurality of hollow needles; and
removing said plurality of hollow needles from said target body surface.

2. The method of claim 1, further comprising the step of subjecting said target body surface to one or more additional treatments selected from the group consisting of galvanic treatment, electrical stimulation, heat treatment and light treatment.

3. The method of claim 2, wherein said one or more additional treatments are provided concurrently with said carboxytherapy using said treatment device.

4. The method of claim 2, wherein said one or more additional treatments are provided prior to or after said carboxytherapy.

5. The method of claim 1, further comprising the step of applying to said target body surface an effective amount of a treatment composition formulized for topical administration.

6. The method of claim 1, wherein said plurality of hollow needles are microneedles having a diameter of about 0.1 mm to about 0.5 mm and a length of about 0.4 mm to about 2.1 mm.

7. The method of claim 1, wherein said target body surface is suffering from a skin condition selected from the group consisting of acne, psoriasis, skin infections, blemishes, hyperpigmentation, hypopigmentation, alopecia, excessive hair growth, unwanted hair growth, rough skin, dry skin, lax skin, wrinkles, hypervasculatated skin, sebum production disorders, excessive pore appearance, excessive perspiration, hyperhidrosis, tattoo appearance, rashes, scar appearance, pain, itch, burn, inflammation, warts, corns, calluses, edema, ivy/oak poisoning, and bites from insects and animals.

8. The method of claim 1, wherein said target body surface is scalp suffering from hair loss.

9. The method of claim 1, wherein said target body surface is suffering from alopecia areata.

10. The method of claim 1, wherein said target body surface is suffering from diabetic ulcer.

11. The method of claim 1, wherein said target body surface is suffering from straie.

12. The method of claim 1, wherein said target body surface is a scarred body surface.

13. A method for treating hair loss in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

14. The method of claim 13, wherein said CO₂ is introduced at a flow rate of 5-300 ml/min.

15. The method of claim 13, wherein said sufficient period of time is 10-60 minutes.

16. The method of claim 13, wherein said introducing step is repeated 4-20 times with an interval of about 24-72 hours between any two repeats.

17. The method of claim 13, further comprising applying a local anesthetic to said target area prior to said introducing step.

18. The method of claim 13, further comprising administering to said target area a composition comprising a hair growth promoting agent, wherein said composition is formulated for topical administration.

19. The method of claim 13, further comprising the step of subjecting said target area to one or more additional treatments selected from the group consisting of galvanic treatment, electrical stimulation, heat treatment and light treatment, wherein said one or more additional treatments are performed concurrently with said introducing step.

20. A method for treating a skin condition in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

21. The method of claim 20, wherein said CO₂ is introduced at a flow rate of 5-360 ml/min.

22. The method of claim 20, wherein said sufficient period of time is 2-120 minutes.

23. The method of claim 20, wherein said introducing step is repeated 1-40 times with an interval of about 6-120 hours between any two repeats.

24. The method of claim 20, further comprising applying a local anesthetic to said target area prior to said introducing step.

25. The method of claim 20, further comprising administering to said target area a composition comprising a therapeutic agent, wherein said composition is formulated for topical administration.

26. The method of claim 20, further comprising the step of subjecting said target area to one or more additional treatments selected from the group consisting of galvanic treatment, electrical stimulation, heat treatment and light treatment, wherein said one or more additional treatments are performed concurrently with said introducing step.

27. A method for promoting wound healing in a target area of a subject, comprising: introducing an effective amount of CO₂ into subcutaneous tissue of said target area for a sufficient period of time, wherein said effective amount of CO₂ is introduced into said target area with a plurality of hollow needles that puncture epidermis of said target area and release CO₂ at a desired rate within said subcutaneous tissue.

28. The method of claim 27, wherein said CO₂ is introduced at a flow rate of 5-300 ml/min.

29. The method of claim 27, wherein said sufficient period of time is 10-60 minutes.

30. The method of claim 27, wherein said introducing step is repeated 5-20 times with an interval of about 24-120 hours between any two repeats.

31. The method of claim 27, further comprising applying a local anesthetic to said target area prior to said introducing step.

32. The method of claim 27, further comprising administering to said target area a composition comprising a wound healing promoting agent, wherein said composition is formulated for topical administration.

33. The method of claim 27, further comprising the step of subjecting said target area to one or more additional treatments selected from the group consisting of galvanic treatment, electrical stimulation, heat treatment and light treatment, wherein said one or more additional treatments are performed concurrently with said introducing step.

34. A disposable skin treatment device, comprising a plurality of hollow needles attached to a surface of a base, wherein said hollow needles having diameters in the range of 0.1-0.5 mm and lengths in the range of 0.4-2.5 mm and wherein said base is attachable to a handheld device to form a fluid communication between at least one of said plurality of hollow needles; wherein at least one of said plurality of hollow needles comprises a sharp end capable of penetrating human epidermis under hand pressure.

35. The disposable skin treatment device of claim 34, wherein said plurality of hollow needles comprise at least 10 needles and wherein said surface of said base has a size of at least 10 cm².

36. The disposable skin treatment device of claim 34, further comprising a housing, wherein said base is attached to said housing and wherein said housing is attachable to a CO₂ source.

37. A method of applying carboxytherapy to a target body surface of a subject in need of such treatment, comprising the steps of:
piercing the skin of said target body surface with a plurality of hollow needles, wherein at least one of said plurality of hollow needles is in fluid communication with a CO₂ source,
injecting a therapeutic amount of CO₂ to said subject through said at least one of said plurality of hollow needles; and
removing said plurality of hollow needles from said skin.

38. The method of claim 37, wherein only the stratum corneum of said skin is pierced.

39. The method of claim 37, wherein the stratum corneum and other layers of the epidermis are pierced.

40. The method of claim 37, wherein said CO₂ is injected at a flow rate of 5-300 ml/min.

41. The method of claim 37, further comprising applying a local anesthetic to said target body surface prior to said piercing step.

42. The method of claim 37, wherein said target body surface is suffering from a skin condition selected from the group consisting of acne, psoriasis, skin infections, blemishes, hyperpigmentation, hypopigmentation, alopecia, excessive hair growth, unwanted hair growth, rough skin, dry skin, lax skin, wrinkles, hypervasculatated skin, sebum production disorders, excessive pore appearance, excessive perspiration, hyperhidrosis, tattoo appearance, rashes, scar appearance, pain, itch, burn, inflammation, warts, corns, calluses, edema, ivy/oak poisoning, and bites from insects and animals.
